SEQUENCE LISTING (20 sequences)

SEQ ID NO: 1 - DNA, 10 bp, Artificial Sequence
  Description of Artificial Sequence Synthetic Primer
aaatcggagc

SEQ ID NO: 2 - DNA, 10 bp, Artificial Sequence
  Description of Artificial Sequence Synthetic Primer
gtcctactcg

SEQ ID NO: 3 - DNA, 10 bp, Artificial Sequence
  Description of Artificial Sequence Synthetic Primer
gtccttagcg

SEQ ID NO: 4 - DNA, 10 bp, Artificial Sequence
  Description of Artificial Sequence Synthetic Primer
tgcgcgatcg

SEQ ID NO: 5 - DNA, 10 bp, Artificial Sequence
  Description of Artificial Sequence Synthetic Primer
aacgtacgcg

SEQ ID NO: 6 - DNA, 10 bp, Artificial Sequence
  Description of Artificial Sequence Synthetic Primer
gcacgccgga

SEQ ID NO: 7 - DNA, 10 bp, Artificial Sequence
  Description of Artificial Sequence Synthetic Primer
caccctgcgc

SEQ ID NO: 8 - DNA, 10 bp, Artificial Sequence
  Description of Artificial Sequence Synthetic Primer
ctatcgccgc

SEQ ID NO: 9 - DNA, 10 bp, Artificial Sequence
  Description of Artificial Sequence Synthetic Primer
cgggatccgc

SEQ ID NO: 10 - DNA, 10 bp, Artificial Sequence
  Description of Artificial Sequence Synthetic Primer
gcgaattccg

SEQ ID NO: 11 - DNA, 10 bp, Artificial Sequence
  Description of Artificial Sequence Synthetic Primer
ccctgcaggc

SEQ ID NO: 12 - DNA, 10 bp, Artificial Sequence
  Description of Artificial Sequence Synthetic Primer
ccaagcttgc

SEQ ID NO: 13 - DNA, 10 bp, Artificial Sequence
  Description of Artificial Sequence Synthetic Primer
gtgcaatgag

SEQ ID NO: 14 - DNA, 10 bp, Artificial Sequence
  Description of Artificial Sequence Synthetic Primer
aggatacgtg

SEQ ID NO: 15 - DNA, 10 bp, Artificial Sequence
  Description of Artificial Sequence Synthetic Primer
aagatagcgg

SEQ ID NO: 16 - DNA, 10 bp, Artificial Sequence
  Description of Artificial Sequence Synthetic Primer
ggatctgaac

SEQ ID NO: 17 - DNA, 10 bp, Artificial Sequence
  Description of Artificial Sequence Synthetic Primer
ttgtctcagg

SEQ ID NO: 18 - DNA, 10 bp, Artificial Sequence
  Description of Artificial Sequence Synthetic Primer
catcccgaac

SEQ ID NO: 19 - DNA, 10 bp, Artificial Sequence
  Description of Artificial Sequence Synthetic Primer
ggactccacg

SEQ ID NO: 20 - DNA, 10 bp, Artificial Sequence
  Description of Artificial Sequence Synthetic Primer
agcctgacgc

(19) United States
(12) Patent Application Publication
(10) Pub. No.: US 2005/0091705 A1
(43) Pub. Date: Apr. 28, 2005

(54) High essential oil and eugenol yielding cultivar of Ocimum sanctum 'CIM-AYU'

(75) Inventors: Raj Kishori Lal, Lucknow (IN); Suman Preet Singh Khanuja, Lucknow (IN); Arun Kumar Agnihotri, Lucknow (IN); Hari Om Misra, Lucknow (IN); Ajit Kumar Shasany, Lucknow (IN); Ali Arif Naqvi, Lucknow (IN); Om Prakash Dhawan, Lucknow (IN); Alok Kalra, Lucknow (IN); Janak Raj Bahl, Lucknow (IN); Mahendra Pandurang Darokar, Lucknow (IN)

Correspondence Address: Bernhard D. Saxe;FOLEY & LARDNER, Washington Harbour, 3000 K Street, N.W., Suite 500, Washington, DC, 20007-5109, US

(73) Assignee: COUNCIL OF SCIENTIFIC AND INDUSTRIAL RESEARCH

(21) Appl. No.: 10/639,682

(22) Filed: Aug. 13, 2003

(51) Int. Cl. A01H 1/00; C12N 15/05

(52) U.S. Cl. 800/277

(57) ABSTRACT

The present invention relates to a high herb yielding essential oil herb variety of Krishna Tulsi (Ocimum sanctum, Family—Lamiaceae, 2n=32) named as ‘CIM-AYU’, more particularly, the invention is related to the development of a high eugenol yielding variety of Krishna Tulsi named ‘CIM-AYU’ through open pollination in the germplasm followed by recurrent progeny selection and evaluation for the yield characters of selected population for 3 years in field conditions, the selected variety is high yielding and stable in subsequent generation. This invention thus relates to the high yielding seeds, plants and plant parts of plant named ‘CIM-AYU’ and its components, to a method of producing named ‘CIM-AYU’, and to a method for producing high eugenol using ‘CIM-AYU’ as a pollinator or parent.

### FIELD OF THE INVENTION

The present invention relates to a high herb yielding essential oil herb variety of Krishna Tulsi (Ocimum sanctum, Family—Lamiaceae, 2n=32) named as ‘CIM-AYU’. More particularly, the invention is related to the development of a high eugenol yielding variety of Krishna Tulsi named ‘CIM-AYU’ through open pollination in the germplasm followed by recurrent progeny selection and evaluation for the yield characters of selected population for 3 years in field conditions. The selected variety is high yielding and stable in subsequent generation. This invention thus relates to the high yielding seeds, plants and plant parts of plant named ‘CIM-AYU’ and its components, to a method of producing named ‘CIM-AYU’, and to a method for producing high eugenol using ‘CIM-AYU’ as a pollinator or parent.

### BACKGROUND OF INVENTION

Tulsi, Ocimum sanctum, belongs to the family of Lamiaceae. Hindus know the plant as Tulasi and Surasah in Sanskrit, and Tulsi in Hindi. Due to its anti oxidant and anti ageing effects Hindus used fresh leaves of this plant daily in his Panchamrut/Charanamrut drink after puja. Other commonly used names are Haripriya, dear to Vishnu, and Bhutagni, destroyer of demons. Tulsi is Divinity. It is regarded not merely as a utilitarian Godsend, as most sacred plants is viewed to be, but as an incarnation of the Goddess Herself. A plethora of Puranic legends and village stories relate how Tulsi came to grow and be worshiped on Earth. The classic Hindu myth, Samudramathana, the “Churning of the Cosmic Ocean,” explains that Vishnu spawned Tulsi from the turbulent seas as a vital aid for all mankind. More common are legends that describe how the Goddess Herself came to reside on Earth as Tulsi.

The tulsi leaf, when eaten, can control thirst, and so was invaluable to weary travellers. Soon, the plant acquired a religious significance, and became essential in worship. Apart from so much religious importance, the Tulsi has immense medicinal properties. It is rich in carbohydrate, fibre, phosphorous, calcium, protein, iron, beta-carotene, vitamins B1 and B2 and in aromatic oils. It is good for colds and coughs, indigestion, stomach pain and diarrhoea. Nausea, ulcers, ringworm and asthma can be effectively treated with Tulsi. It is said to lower blood sugar and increase lactation.

The oil is used in anti-perspirants and in fly and mosquito repellents. Keeping in mind the importance of tulsi, the need for developing a better plant type having high herb, essential yield characters combined with consistent high yield of phenyl propanoid “Eugenol” in the essential oil in all around the year was felt and planned breeding and selection process was undertaken at the farm of CIMAP, Lucknow to develop the variety CIM-AYU. Generally in Indian condition the high eugenol yielding genotypes do not produce eugenol during rainy season. Therefore, the primary objective of the present invention was to develop a new Ocimum santlum cultivar possessing a high yield of essential oils especially Eugenol, during the rainy season. To develop such a high yielding cultivar, recurrent selection was undertaken for the genotypes producing higher eugenol during the rainy season. Further to establish high yielding essential oil characteristics recurrent backcrossing and selection were undertaken which finally resulted in a plant cultivar named ‘CIM-AYU’ of O. santalum. This plant developed produced high amount of essential oils especially eugenol during the rainy season. Thus during the process of selection emphasis was on selecting a genotype producing high eugenol during rainy season which resulted in a plant named ‘CIM-AYU’.

### DETAILED DESCRIPTION

Breeding History

Mass and recurrent selections can be used to improve populations of either self- or cross-pollinating crops. A genetically variable population of heterozygous individuals is either identified or created by inter-crossing several different parents. The best plants are selected based on individual superiority, outstanding progeny, or excellent combining ability. The selected plants are inter-crossed to produce a new population in which further cycles of selection are continued.

Selection for three years: In the present invention which was carried out under genetic improvement programme of Tulsi (Ocimum sanctum), the diverse germplasm/genetic stocks of Krishana tulsi were collected from Utter Pradesh, Bihar, Madhya Pradesh, Orissa, Uttranchal, Gujarat, Maharashtra, Jammu and Kashmir, West Bangal states of India. The germplasm were grown in plots of 1.0 m×10.0 m size plots with 50 cm×50 cm spacing randomly. The seeds collected from individual plants were bulked and germinated in the next season (May) and transferred to the main field for evaluation (in June). Randomly 2500 plants were evaluated for better plant types with high herb and oil yield, out of which 10 plants were selected. The seeds from these plants were again bulked and planted in the next season out of which 10 plants selected again. The same process was repeated in the next year and ultimately 10-plant types were taken for preliminary evaluation.

Evaluation trials: Ten promising genetic stocks along with a check (control) STA-1 were evaluated in Initial Evaluation Trial (IET). The evaluations were carried out in randomised block design (RBD) with two replications in 0.50 m² plots for each treatment during year 2000-2001. Four best performing selections KRT-2, SHTS-3, OTF-4, STN-5 from IET and one check STA-1 were evaluated in Bench Scale Trial (BST). The evaluations were carried out in randomised block design (RBD) with four replications in 12.24 m² plots for each treatment during year 2001-2002. Finally, three most promising selections KRT-2, STAS-3 and STN-5 along with a check STA-1 were evaluated in Pilot Scale Trial (PST) in 2002-2003 (84 m² for each treatment) (Table 1). On an average, the elite strain KRT-2 registered its superiority over all other selections including check STA-1 for whole plant herb, dry leaves and oil yield of better quality per unit area (Table 2). The elite strain was named as variety CIM-AYU.

During screening and experimentation individual strains were maintained in seed plots with an isolation distance of 500 m² and seeds obtained from these seed plots were used in growing the plants for evaluation. During the evaluation trials 10 plants from each strains were evaluated through profiling the population DNA and comparing among each other for maintenance of purity and stability through generations. The plants when grown in isolation as mentioned are self pollinated and maintain the stability and purity as observed from the morphological, essential oil and DNA profiles.

### DETAILED DESCRIPTION OF THE INVENTION

According the present invention relates to a strain KRT-2, subsequently has been named as ‘CIM-AYU’, thus the developed plant variety is named as ‘CIM-AYU’, which consistently showed higher herbage and oil content in BST and PST. The yield of the complete plant was estimated to be 200 quintals per hectare with dry leaf yield of 15.85 quintals per hectare. The total oil yield was 110.95 kilogram per hectare. All the yields (total plant, dry leaf and oil) were higher than all other strains taken for comparison. The variety CIM-AYU produced higher eugenol in the essential oil compared to the control variety. In Indian agro-climatic condition during rainy season it is observed that the eugenol content of the leaf decrease drastically in many of the Ocimum sanctum genotypes tending sometimes to zero from 80%. But in this variety during rainy season the eugenol content was found to be 47% and this was observed during three consecutive growing seasons, for three consecutive years [Photograph 1].

The main embodiment of the present invention relates to a new and distinct high yielding essential oil plant of Ocimum santlum of variety Tulsi Krishna named ‘CIM-AYU’ developed through cross open pollination of diversely collected germplasm followed by recurrent progeny selection and evaluation by screening for morphology, high herbal and essential oil content and further recurrent selection for uniformity and stability for a period of three years to establish selected traits such as high herbal and essential oil content, the said plant after yield evaluation in the field having following characteristics:
a. the said plant produces a higher amount of eugenol (83% of oil) with high essential oil yield (0.7 to 0.72%) as well as herbage yield (at least fresh total plant 200 and dry leaf 15.85 quintals per hectare),
b. the said plant possesses better growth and vegetative growth with 66 cm×78 cm canopy area and a height of at least 75.67 cm in a maximum of 100 days,
c. the said plant is erect sturdy with profuse synchronous branching in growth habit
d. the said plant produces stem which is a round to quadrangular, woody, green (139D), faint purplish red pigmentation at time of maturity (79D), with mean thickness of 0.7 cm.
e. the said plant has distinct molecular profile by random amplified polymorphic DNA (RAPD) using 12 random primers distinguishing the plant from the other existing varieties
f. the said plant produces leaves having light greenish (137C), chartaceous texture, minute surface hairs on the above, hirsute hairs below with nerves, oblong to ob-lanceolate shape with serrate marigins, sub-acute to acuminate tip, obtuse base and moderately broad size
g. said plant produces a petiole extending to size of 2.4 cm
h. said plant spreads in an area of 15.8 cm², at average of full branch
i. the said plant produces an indefinite racemose inflorescence with 10-15 florets having light green colour (142B) in early stage and pale purple when mature (79D) with flowers arranged in whorls surrounding the peduncle at the base of the lateral leaves having pinkish white flowers (56C)
j. the said plant is able to produce higher herbage, oil and eugenol yield per unit area as compared to other control genotypes
k. the said plant produces full plant herb yield 200 quintal per hectare and from fresh leaves yield 63.39 ql per hectare to 15.85 quintal per hectare dry leave yield
l. the said plant produces at least 110 kilogram per hectare essential oil rich in eugenol (80 to 84%) the content during the growing season.
m. the said plant produces eugenol in the essential oil even during rainy season (at least 47%) compared to 5.0% in check and other strains.
   Uniformity and Stability

When the uniform plant population of CIM-AYU is grown in isolation with minimum isolation distance of 500 m from other genotypes of Ocimum sanctum, outcrossing from undesirable genotypes does not take place. Instead the population purity is being maintained through restricting pollination within the population components maintain allelic balance with in the population in nature of equilibrium. In the present invention the purity of the plant variety was maintained by growing the plant population with an isolation distance of 500 m from any other genotypes of Ocimum sanctum. The stability of the plant population was checked through pooled DNA profiling using 20 MAP primers in subsequent generations and found to be uniform without variation. The sequences of the primers MAP01 to MAP20 were AAATCGGAGC, GTCCTACTCG, GTCCTTAGCG, TGCGCGATCG, AACGTACGCG, GCACGCCGGA, CACCCTGCGC, CTATCGCCGC, CGGGATCCGC, GCGAATTCCG, CCCTGCAGGC, CCAAGCTTGC, GTGCAATGAG, AGGATACGTG, AAGATAGCGG, GGATCTGAAC, TTGTCTCAGG, CATCCCGAAC, GGACTCCACG, AGCCTGACGC, respectively. The primers AAATCGGAGC, TGCGCGATCG, AACGTACGCG, CGGGATCCGC, GCGAATTCCG, CCCTGCAGGC, CCAAGCTTGC, AAGATAGCGG, GGATCTGAAC, TTGTCTCAGG, GGACTCCACG, CACCCTGCGC were used to develop the unique fingerprint pattern of the variety and the pattern was found to be consistent for three generations. These latter primers give a specific and unique DNA fingerprinting pattern for the plant named ‘CIM-AYU’. These primers therefore can be considered as the cultivar CIM-AYU specific for they represent the DNA patterns found only for in this plant cultivar [Photograph 2].

### BRIEF DESCRIPTION OF THE ACCOMPANYING DRAWINGS

FIG. 1: shows photograph of the plant of CIM-AYU.

FIG. 2: shows Lane: 1 Hind III digest molecular weight markers: Lane 2 to 13: Profiles with AAATCGGAGC, TGCGCGATCG, AACGTACGCG, CGGGATCCGC, GCGAATTCCG, CCCTGCAGGC, CCAAGCTTGC, AAGATAGCGG, GGATCTGAAC, TTGTCTCAGG, GGACTCCACG, CACCCTGCGC primers.

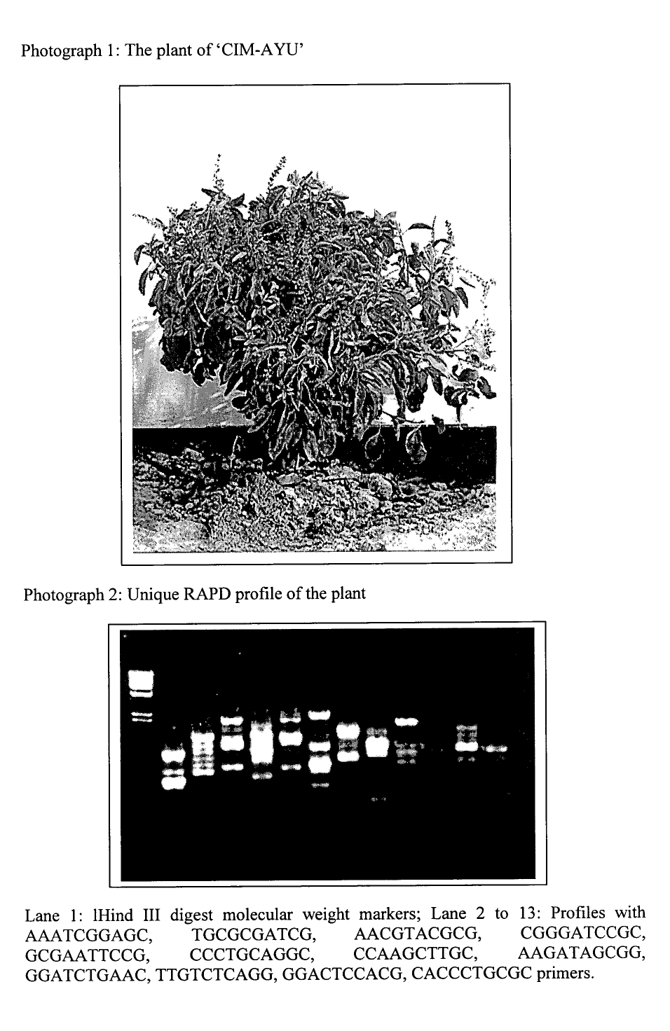

TABLE 1
Mean performance of promising strains in different yield trials for herb and oil yield in Ocimum sanctum

| | | BST (RBD, reps 3, plot size-12.24 sqm) | | | | | PST (plot size-84.0 sqm) | | | | | | |
|---|---|---|---|---|---|---|---|---|---|---|---|---|---|
| | | | | | Oil | Oil | | | | Oil | | | Oil |
| | | Mean herb yield | | | content | yield | Mean herb yield | | | content | | | yield |
| | | (kg/plot) | | | (%) | (g/plot) | (kg/plot) | | | (%) | Herb yield (ql/ha) | | (kg/ha) |
| S. | | Complete | Fresh | Dry | Dry | Dry | Complete | Fresh | Dry | Dry | Complete | Dry | Dry |
| No | Entries | plant | leaves | leaves | leaves | leaves | plant | leaves | leaves | leaves | plant | leaves | leaves |
| 1. | KRT-2 | 34.20 | 9.15 | 2.31 | 0.705 | 16.27 | 168.00 | 53.25 | 13.31 | 0.70 | 200.00 | 15.85 | 110.95 |
| 2. | STAS-3 | 34.31 | 8.99 | 1.99 | 0.628 | 12.45 | 152.04 | 55.87 | 12.04 | 0.64 | 181.00 | 14.33 | 91.71 |
| 3. | OTF-4 | 11.63 | 3.71 | 0.92 | 0.383 | 3.50 | — | — | — | — | — | — | — |
| 4. | STN-5 | 16.74 | 6.72 | 1.48 | 0.478 | 7.04 | 113.39 | 46.20 | 10.84 | 0.40 | 134.99 | 12.90 | 51.0 |
| 5. | STA-1 | 12.76 | 3.51 | 0.89 | 0.225 | 2.01 | 113.40 | 42.42 | 10.92 | 0.23 | 135.00 | 13.00 | 29.90 |
| | (CHECK) | | | | | | | | | | | | |
| | CD_{(5%)} | 0.39 | 0.08 | 0.04 | 0.034 | 0.51 | — | — | — | — | — | — | — |
| | CD_{(1%)} | 0.55 | 0.11 | 0.05 | 0.048 | 0.71 | — | — | — | — | — | — | — |

TABLE 2
Oil composition of variety CIM-AYU (KRT-2) and
check STA-1 of Ocimum sanctum

| S. No. | Constituents in oil | CIM-AYU (KRT-2) | STA-1 (Check) |
|---|---|---|---|
| 1. | β-pinene | 0.13 | — |
| 2. | α-pinene | 0.19 | 0.05 |
| 3. | Camphene | 0.09 | 0.08 |
| 4. | Sabinene | 0.03 | 0.13 |
| 5. | Limonene | 0.05 | 0.78 |
| 6. | p-Cymene | — | 0.11 |
| 7. | Cineole | 0.21 | 2.50 |
| 8. | Linalool | 0.05 | 1.92 |
| 9. | Eugenol | 83.56 | 40.42 |
| 10. | β-elemene | 7.47 | 14.11 |
| 11. | β-caryophyllene | 6.93 | 9.07 |
| 12. | Germacrene-D | — | 16.65 |

Taxonomic Description of the Ocimum Plant Named ‘CIM-AYU’

| 1. | Genus: Ocimum | |
|---|---|---|
| 2. | Species: sanctum L. | |
| 3. | Family: Lamiaceae | |
| 4. | Common name: Krishna Tulsi | |
| 5. | Plant height: 75.67 cm in 100 days | |
| 6 | Plant canopy: 66 cm × 78 cm | |
| 7. | Growth habit: Erect sturdy main stem, profuse synchronous | |
| | branching. | |
| 8. | Stem: Round to quadrangular, woody, green (139D), faint purplish | |
| | red pigmentation at the time of maturity (79D), Mean thickness | |
| | 0.7 cm. | |
| 9. | Leaf: | |
| | Colour | Light green (137C) both upper and lower surface |
| | Texture | Chartaceous |
| | Surface | Minute hairs above, hirsute below and nerves |
| | Shape | Oblong to ob-lanceolate |
| | Margin | Serrate |
| | Tip | Sub-acute to acuminate |
| | Base | Obtuse |
| | Size | Moderately broad |
| | Petiole length | 2.4 cm |
| | Area | 15.8 cm² (Average of full branch) |
| | Length | 4.93 cm |
| | Width | 2.73 cm |

| 10. | Leaf: stem ratio (w/w): 0.84 | |
|---|---|---|
| 11. | Inflorescence: Indefinite recemose | |
| | Total number of florets 10 to 15 | |
| | colour early stage light green (142B) | |
| | Colour maturity Pale purple (79D) | |
| 12. | Flowers | |
| | Arranged in whorls surrounding the peduncle at the base | |
| | of lateral leaves. | |
| 13. | Oil content in the dry leaf (%): 0.7 to 0.72 | |
| 14. | Oil quality: | |
| | Eugenol (%): 80 to 84 | |
| 15. | Herbage (Quintal per hectare): | Fresh total plant: 200 |
| | | Dry leaf: 15.85 |

Comparison of Character of CIM-AYU with the Check (STA-1)

| | Elite strain | Check |
|---|---|---|
| Attributes | CIM-AYU/KRT-2 | STA-1 |
| Plant height (cm) | 75.67 | 63.33 |
| Days to flowering (50%) | 76 (early) | 92 (late) |
| Growth habit | Semi closed | open |
| No. of branches/plant | 17.33 | 12.67 |
| Length of leave (cm) | 4.93 | 4.63 |
| Width of leave (cm) | 2.73 | 2.83 |
| Whole plant herb yield (ql/ha) | 200.00 | 135.00 |
| Dry leaf yield (q/ha) | 15.85 | 13.00 |
| Oil content (%) | 0.70 | 0.23 |
| Oil yield (Kg/ha) | 110.95 | 29.90 |
| Colour of stem | light green | Violet |
| Colour of leaves | light green | light Violet |
| Eugenol (%) | 83.56 | 40.41 |
| β-Elemene (%) | 7.47 | 14.11 |
| β-Caryophyllene (%) | 6.93 | 9.07 |

Time and Place of Development of the Variety

The variety CIM-AYU was developed at CIMAP and all the field trials were conducted at the farm of CIMAP, Lucknow. The selection and screening was started in the year 1997 and from the year 2000 the yield trials were undertaken till 2002.

1. A new and distinct high yielding essential oil plant of Ocimum santlum of variety Tulsi Krishna named ‘CIM-AYU’ developed through cross open pollination of diversely collected germplasm followed by recurrent progeny selection and evaluation by screening for morphology, high herbal and essential oil content and further recurrent selection for uniformity and stability for a period of three years to establish selected traits such as high herbal and essential oil content, the said plant after yield evaluation in the field having following characteristics:
(a) the said plant produces a higher amount of eugenol (83% of oil) with high essential oil yield (0.7 to 0.72%) as well as herbage yield (at least fresh total plant 200 and dry leaf 15.85 quintals per hectare),
(b) the said plant possesses better growth and vegetative growth with 66 cm×78 cm canopy area and a height of at least 75.67 cm in a maximum of 100 days,
(c) the said plant is erect sturdy with profuse synchronous branching in growth habit,
(d) the said plant produces stem which is a round to quadrangular, woody, green (139D), faint purplish red pigmentation at time of maturity (79D), with mean thickness of 0.7 cm,
(e) the said plant has distinct molecular profile by random amplified polymorphic DNA (RAPD) using 12 random primers distinguishing the plant from the other existing varieties,
(f) the said plant produces leaves having light greenish (137C), chartaceous texture, minute surface hairs on the above, hirsute hairs below with nerves, oblong to ob-lanceolate shape with serrate marigins, sub-acute to acuminate tip, obtuse base and moderately broad size,
(g) said plant produces a petiole extending to size of 2.4 cm,
(h) said plant spreads in an area of 15.8 cm², at average of full branch,
(i) the said plant produces an indefinite racemose inflorescence with 10-15 florets having light green colour (142B) in early stage and pale purple when mature (79D) with flowers arranged in whorls surrounding the peduncle at the base of the lateral leaves having pinkish white flowers (56C),
(j) the said plant is able to produce higher herbage, oil and eugenol yield per unit area as compared to other control genotypes,
(k) the said plant produces full plant herb yield 200 quintal per hectare and from fresh leaves yield 63.39 quintal per hectare to 15.85 quintal per hectare dry leave yield,
(l) the said plant produces at least 110 kilogram per hectare essential oil rich in eugenol (80 to 84%) the content during the growing season, and
(m) the said plant produces eugenol in the essential oil even during rainy season (at least 47%) compared to 5.0% in check and other strains.
